(19) United States
(12) Patent Application Publication
(10) Pub. No.: US 2002/0173502 A1
(43) Pub. Date: Nov. 21, 2002

(54) Pharmaceutically active compounds

(76) Inventor: Charlotte Moira Norfor Allerton, Sandwich (GB)

Correspondence Address: Gregg C. Benson, Pfizer Inc., Patent Department, MS 4159, Eastern Point Road, Groton, CT, 06340, US

(21) Appl. No.: 10/092,992

(22) Filed: Mar. 6, 2002

Related U.S. Application Data
(60) Provisional application No. 60/291,714, filed on May 17, 2001.

(30) Foreign Application Priority Data
Mar. 9, 2001 (GB) 0105893.2

(51) Int. Cl. A61K 31/519; C07D 487/02

(52) U.S. Cl. 514/210.21; 544/262

(57) ABSTRACT

There is provided a general formula I:

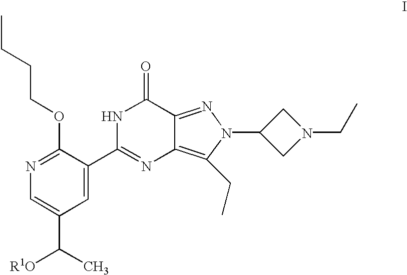

or a pharmaceutically or veterinarily acceptable salt or polymorph and/or solvate thereof, wherein

R¹ represents H; C(O)C₁-C₄ alkyl; C(O)aryl; C(O)heteroaryl.

and which is useful in the curative and prophylactic treatment of a medical condition for which inhibition of a cyclic guanosine 3′,5′-monophosphate phosphodiesterase (and in particular cGMP PDE5) is desired.

### FIELD OF THE INVENTION

This invention relates to pharmaceutically useful compounds, and in particular to 5-[2-butoxy-5-(1-hydroxyethyl)-3-pyridinyl]-3-ethyl-2-(1-ethyl-3-azetidinyl)-2,6-dihydro-7H-pyrazolo[4,3-d]pyrimidin-7-one, which are useful in the inhibition of cyclic guanosine 3′,5′-monophosphate phosphodiesterases (cGMP PDEs), such as type 5 cyclic guanosine 3′,5′-monophosphate phosphodiesterases (cGMP PDE5). The compounds therefore have utility in a variety of therapeutic areas, including male erectile dysfunction (MED).

### PRIOR ART

International application WO 01/27112 discloses the use of pyrazolopyrimidinone compounds having pyridinyl functionality with 5′ substitution. A particularly preferred compound in WO 01/27112 is 5-(5-acetyl-2-butoxy-3-pyridinyl)-3-ethyl-2-(1-ethyl-2-(1-ethyl-3-azetidinyl)-2,6-dihydro-7H-pyrazolo[4,3-d]pyrimidin-7-one (the compound of Preparation 1 herein).

### DISCLOSURE OF THE INVENTION

According to the present invention, there is provided a compound of general formula I:

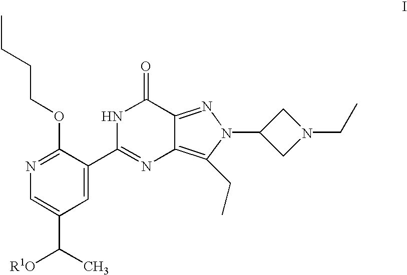

or a pharmaceutically or veterinarily acceptable salt or polymorph and/or solvate thereof, wherein

R¹ represents H; C(O)C₁-C₄ alkyl; C(O)aryl; C(O)heteroaryl.

which compounds are referred to together hereinafter as “the compounds of the invention”.

The term C₁-C₄ alkyl includes methyl, ethyl, propyl and butyl groups. Unless otherwise specified, such alkyl groups may, when there is a sufficient number of carbon atoms, be linear or branched, be saturated or unsaturated, be cyclic, acyclic or part cyclic/acyclic, and/or be substituted and/or terminated by one or more halo atoms. Preferred C₁-C₄ alkyl groups for use herein are C₁-₃ alkyl groups.

The term “aryl”, when used herein, includes six- to ten-membered carbocyclic aromatic groups, such as phenyl and naphthyl, which groups may include fused rings and which groups are optionally substituted with one or more substituents selected from aryl (which group may not be substituted by any further aryl groups), C₁-C₆ alkyl, Het, halo, CN, nitro, OR², OC(O)R², C(O)R², C(O)OR², C(O)NR²R³, NR²R³ and SO₂NR²R³ wherein R² and R³ may independently represent H or C₁-C₄ alkyl, preferably H or methyl or ethyl.

The term “Het”, when used herein, includes four- to twelve-membered, preferably four- to ten-membered, ring systems, which rings contain one or more heteroatoms selected from nitrogen, oxygen, sulphur and mixtures thereof, and which rings may contain one or more double bonds or be non-aromatic, partly aromatic or wholly aromatic in character. The ring systems may be monocyclic, bicyclic or fused. Each “Het” group identified herein is optionally substituted by one or more substituents selected from halo, cyano, nitro, oxo, C₁-C₆ alkyl (which alkyl group may itself be optionally substituted or terminated as defined below), OR², OC(O)R², C(O)R², C(O)OR², C(O)NR²R³, NR²R³ and SO₂NR²R³ wherein R² and R³ are as hereinbefore defined. The term thus includes groups such as optionally substituted azetidinyl, pyrrolidinyl, imidazolyl, indolyl, furanyl, oxazolyl, isoxazolyl, oxadiazolyl, thiazolyl, thiadiazolyl, triazolyl, tetrazolyl, oxatriazolyl, thiatriazolyl, pyridazinyl, morpholinyl, pyrimidinyl, pyrazinyl, pyridinyl, quinolinyl, isoquinolinyl, piperidinyl, pyrazolyl imidazopyridinyl and piperazinyl. Substitution at Het may be at a carbon atom of the Het ring or, where appropriate, at one or more of the heteroatoms.

“Het” groups may also be in the form of an N-oxide.

Halo groups with which the above-mentioned groups may be substituted or terminated include fluoro, chloro, bromo and iodo.

The pharmaceutically or veterinarily acceptable salts of the compounds of the invention which contain a basic centre are, for example, non-toxic acid addition salts formed with inorganic acids such as hydrochloric, hydrobromic, hydroiodic, sulphuric and phosphoric acid, with carboxylic acids or with organo-sulphonic acids. Examples include the HCl, HBr, HI, sulphate or bisulphate, nitrate, phosphate or hydrogen phosphate, acetate, benzoate, succinate, saccarate, fumarate, maleate, lactate, citrate, tartrate, gluconate, camsylate, methanesulphonate, ethanesulphonate, benzenesulphonate, p-toluenesulphonate and pamoate salts. Compounds of the invention can also provide pharmaceutically or veterinarily acceptable metal salts, in particular non-toxic alkali and alkaline earth metal salts, with bases. Examples include the sodium, potassium, aluminium, calcium, magnesium, zinc and diethanolamine salts. For a review on suitable pharmaceutical salts see Berge et al, J. Pharm, Sci., 66, 1-19, 1977.

The pharmaceutically acceptable solvates of the compounds of the invention include the hydrates thereof.

Also included within the scope of the compound and various salts of the invention are polymorphs thereof.

A compound of the formula (I) contains one or more asymmetric carbon atoms and therefore exists in two or more stereoisomeric forms. The present invention includes the individual stereoisomers of the compounds of the formula (I) and, where appropriate, the individual tautomeric forms thereof, together with mixtures thereof. Separation of diastereoisomers or cis and trans isomers may be achieved by conventional techniques, e.g. by fractional crystallisation, chromatography or H.P.L.C. of a stereoisomeric mixture of a compound of the formula (I) or a suitable salt or derivative thereof. An individual enantiomer of a compound of the formula (I) may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

All stereoisomers are included within the scope of the invention.

The compounds of the invention may exhibit tautomerism. All tautomeric forms of the compounds of formula I, are included within the scope of the invention.

Also included within the scope of the invention are radiolabelled derivatives of compounds of formula I which are suitable for biological studies.

A preferred group of compounds according to a further aspect of the invention, are compounds of formulae I as hereinbefore defined, wherein:

R¹ represents H or C(O)C₁-C₃ alkyl

Particularly preferred herein is compound IA:

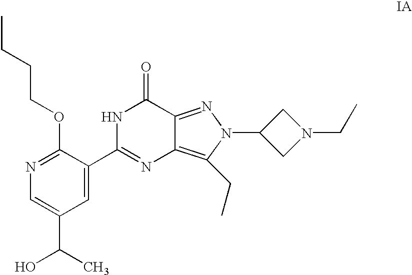

5-[2-butoxy-5-(1-hydroxyethyl)-3-pyridinyl]-3-ethyl-2-(1-ethyl-3-azetidinyl)-2,6-dihydro-7H-pyrazolo[4,3-d]pyrimidin-7-one.

Preparation

The compounds of the invention may be prepared according to the processes exemplified hereinafter.

Specifically compounds of the invention may be prepared via an analogous reaction sequence to that exemplified herein for compound 1A, such analogous reaction sequence can start from the compounds of preparations 4(g).

Substituents on the aryl and Het groups in the above-mentioned compounds may be introduced, and interconverted, using techniques which are well known to those skilled in the art.

The skilled person will also appreciate that various standard substituent or functional group interconversions and transformations within certain compounds of formulae I will provide other compounds of formulae I. For example, when R¹ is CH₃C(O)O— the compounds of formulae I, in which R¹ is H may be formed by hydrolysis with an excess of a suitable acid. Further examples when R¹ is C(O)C₁-C₄alkyl include trans-esterification.

The compounds of the invention may be isolated from their reaction mixtures using conventional techniques.

It will be appreciated by those skilled in the art that, in the course of carrying out the above processes described above, the functional groups of intermediate compounds may need to be protected by protecting groups.

Functional groups which it is desirable to protect include hydroxy, amino and carboxylic acid. Suitable protecting groups for hydroxy include trialkylsilyl and diarylalkylsilyl groups (e.g. tert-butyldimethylsilyl, tert-butyidiphenylsilyl or trimethylsilyl) and tetrahydropyranyl. Suitable protecting groups for amino include tert-butyloxycarbonyl, 9-fluorenyl-methoxycarbonyl or benzyloxycarbonyl. Suitable protecting groups for carboxylic acid include C₁₋₆ alkyl or benzyl esters.

The protection and deprotection of functional groups may take place before or after any of the reaction steps described hereinbefore or after.

Protecting groups may be removed in accordance with techniques which are well known to those skilled in the art.

The use of protecting groups is fully described in “Protective Groups in Organic Chemistry”, edited by J W F McOmie, Plenum Press (1973), and “Protective Groups in Organic Synthesis”, 2^{nd} edition, T W Greene & P G M Wutz, Wiley-Interscience (1991).

Persons skilled in the art will also appreciate that, in order to obtain compounds of formula I in an alternative, and, on some occasions, more convenient manner, the individual process steps mentioned hereinafter may be performed in a different order, and/or the individual reactions may be performed at a different stage in the overall route (i.e. substituents may be added to and/or chemical transformations performed upon, different intermediates to those mentioned hereinbefore in conjunction with a particular reaction). This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis.

Pharmaceutically acceptable acid addition salts of the compounds of formulae I or 1A which contain a basic centre may be prepared in a conventional manner. For example, a solution of the free base may be treated with the appropriate acid, either neat or in a suitable solvent, and the resulting salt may then be isolated either by filtration of by evaporation under vacuum of the reaction solvent. Pharmaceutically acceptable base addition salts can be obtained in an analogous manner by treating a solution of a compound of formula I or IA with the appropriate base. Both types of salt may be formed or interconverted using ion-exchange resin techniques.

The present invention also includes all suitable isotopic variations of a compound of the formula I or IA or a pharmaceutically acceptable salt thereof. An isotopic variation of a compound of the formula I or IA or a pharmaceutically acceptable salt thereof is defined as one in which at least one atom is replaced by an atom having the same atomic number but an atomic mass different from the atomic mass usually found in nature. Examples of isotopes that can be incorporated into compounds of the formula I or IA and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compounds of the formula I or IA and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of formula I or IA and pharmaceutically acceptable salts thereof of this invention can generally be prepared by conventional procedures such as by the illustrative methods or by the preparations described in the Examples and Preparations hereafter using appropriate isotopic variations of suitable reagents.

It will be appreciated by those skilled in the art that certain protected derivatives of compounds of formula I or IA, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of formula I may act as prodrugs of other compounds of formula I.

All protected derivatives, and prodrugs, of compounds of formula I are included within the scope of the invention.

The present invention additionally comprises the combination of a compound according to the present invention, wherein said combination can be administered by sequential, simultaneous or joint administration of a compound of general formula I with:

(1) one or more naturally occurring or synthetic prostaglandins or esters thereof. Suitable prostaglandins for use herein include compounds such as alprostadil, prostaglandin E₁,prostaglandin E_{0,} 13, 14-dihydroprostaglandin E₁, prostaglandin E₂, eprostinol, natural synthetic and semi-synthetic prostaglandins and derivatives thereof including those described in U.S. Pat. No. 6,037,346 issued on Mar. 14, 2000 and incorporated herein by reference, PGE₀, PGE₁, PGA₁, PGB₁, PGF₁α, 19-hydroxy PGA₁, 19-hydroxy - PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, suiprostune, tiaprost and moxisylate; and/or

(2) one or more α-adrenergic receptor antagonist compounds also known as (α-adrenoceptors or α-receptors or α-blockers. Suitable compounds for use herein include: the α-adrenergic receptors as described in PCT application WO99/30697 published on Jun. 14, 1998, the disclosures of which relating to α-adrenergic receptors are incorporated herein by reference and include, selective α₁-adrenoceptors or α₂-adrenoceptors and non-selective adrenoceptors, suitable α₁-adrenoceptors include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blockers from U.S. Pat. No. 6,037,346 [Mar. 14, 2000] dibenarnine, tolazoline, trimazosin and dibenarnine; α-adrenergic receptors as described in U.S. Pat. Nos.: 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptors include: clonidine, papaverine, papaverine hydrochloride, optionally in the presence of a cariotonic agent such as pirxamine; and/or

(3) one or more NO-donor (NO-agonist) compounds. Suitable NO-donor compounds for use herein include organic nitrates, such as mono- di or tri-nitrates or organic nitrate esters including glyceryl brinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075 and/or

(4) one or more potassium channel openers. Suitable potassium channel openers for use herein include nicorandil, cromokalim, levcromakalim, lemakalim, pinacidil, cliazoxide, minoxidil, charybdotoxin, glyburide, 4-amini pyridine, BaCl₂; and/or

(5) one or more dopaminergic agents, preferably apomorphine or a selective D2, D3 or D2/D3 agonist such as pramipexol and ropirinol (as claimed in WO 0023056), L-Dopa or carbi dopa, PNU 95666 (as claimed in WO 00 40226); and/or

(6) one or more vasodilator agents. Suitable vasodilator agents for use herein include nimodepine, pinacidil, cyclandelate, isoxsuprine, chloroprumazine, halo peridol, Rec 15/2739, trazodone; and/or

(7) one or more thromboxane A2 agonists; and/or

(8) one or more ergot alkoloids; Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride; and/or

(9) one or more compounds which modulate the action of atrial natruretic factor (also known as atrial naturetic peptide), B and C type naturetic factors such as inhibitors or neutral endopeptidase; and/or

(10) one or more compounds which inhibit angiotensin-converting enzyme such as enapril, and one or more combined inhibitors of angiotensin-converting enzyme and neutral endopeptidase such as omapatrilat; and/or

(11) one or more angiotensin receptor antagonists such as losartan; and/or

(12) one or more substrates for NO-synthase, such as L-arginine; and/or

(13) one or more calcium channel blockers such as amlodipine; and/or

(14) one or more antagonists of endothelin receptors and inhibitors or endothelin-converting enzyme; and/or

(15) one or more cholesterol lowering agents such as statins (e.g. atorvastatin/Lipitor-trade mark) and fibrates; and/or

(16) one or more antiplatelet and antithrombotic agents, e.g. tPA, uPA, warfarin, hirudin and other thrombin inhibitors, heparin, thromboplastin activating factor inhibitors; and/or

(17) one or more insulin sensitising agents such as rezulin and hypoglycaemic agents such as glipizide; and/or

(18) one or more COX 2 inhibitors; and/or

(19) pregabalene; and/or

(20) gabapentene; and/or

(21) one or more acetylcholinesterase inhibitors such as donezipil; and/or

(22) one or more steroidal anti-inflammatory agents; and/or

(23) one or more estrogen agonists and/or estrogen antagonists, preferably raloxifene or lasofoxifene, (-)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656.

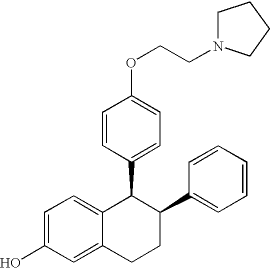

Compound A

(24) one or more one or more of a further PDE inhibitor, more particularly a PDE 2, 4, 7 or 8 inhibitor, preferably PDE2 inhibitor, said inhibitors preferably having an IC50 against the respective enzyme of less than 100 nM: and/or

(25) one or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM , more preferably less than 50nM, suitable NPY and in particular NPY1 inhibitor compounds are described in EP-A-1097718; and/or

(26) one or more of vasoactive intestinal peptide (VIP), VIP mimetic, more particularly mediated by one or more of the VIP receptor subtypes VPAC1,VPAC or PACAP (pituitary adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil); and/or

(27) one or more of a melanocortin receptor agonist or modulator or melanocortin ehancer, such as melanotan II, PT-14, PT-141 or compounds claimed in WO-09964002, WO-00074679, WO-09955679, WO-00105401, WO-00058361, WO-00114879, WO-00113112, WO-09954358; and/or

(28) one or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993; and/or

(29) one or more of a modulator of transporters for noradrenaline, dopamine and/or serotonin, such as bupropion, GW-320659; and/or

(30) one or more of a purinergic receptor agonist and/or modulator; and/or

(31) one or more of a neurokinin (NK) receptor antagonist, including those described in WO-09964008; and/or

(32) one or more of an opioid receptor agonist, antagonist or modulator, preferably agonists for the ORL-1 receptor; and/or

(33) one or more of an agonist or modulator for oxytocin/vasopressin receptors, preferably a selective oxytocin agonist or modulator; and/or

(34) one or more modulators of cannabinoid receptors; and/or

(35) one or more of an NEP inhibitor, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719; and/or

(36) one or more compounds which inhibit angiotensin-converting enzyme such as enalapril, and one or more combined inhibitors of angiotensin-converting enzyme and neutral endopeptidase such as omapatrilat; and/or

(37) one or more tricyclic antidepressants, e.g. amitriptiline; and/or

(38) one or more non-steroidal anti-inflammatory agents; and/or

(39) one or more angiotensin-converting enzyme (ACE) inhibitors, e.g. quinapril; and/or

(40) one or more anti-depressants (such as clomipramine and SSRIs (such as paroxetine and sertaline).

wherein said combination can be in the form of co-administration, simultaneous administration, concurrent administration, or stepwise administration.

Medical Use

The compounds of the invention are useful because they possess pharmacological activity in animals, especially mammals, including humans. They are therefore indicated as pharmaceuticals, as well as for use as animal medicaments.

According to a further aspect of the invention there is provided the compounds of the invention for use as pharmaceuticals, and for use as animal medicaments.

In particular, compounds of the invention have been found to be inhibitors of cGMP PDEs, such as cGMP PDE5, for example as demonstrated in the tests described below, and are thus useful in the treatment of medical conditions in humans, and in animals, in which cGMP PDEs, such as cGMP PDE5, are indicated, and in which inhibition of cGMP PDEs, such as cGMP PDE5, is desirable.

By the term “treatment”, we include both therapeutic (curative), palliative or prophylactic treatment.

Thus, according to a further aspect of the invention there is provided the use of the compounds of the invention in the manufacture of a medicament for the treatment of a medical condition in which a cGMP PDE (e.g. cGMP PDE5) is indicated. There is further provided the use of the compounds of the invention in the manufacture of a medicament for the treatment of a medical condition in which inhibition of a cGMP PDE (e.g. cGMP PDE5) is desirable.

The compounds of the invention are thus expected to be useful for the curative, palliative or prophylactic treatment of mammalian sexual disorders. In particular, the compounds are of value in the treatment of mammalian sexual dysfunctions such as male erectile dysfunction (MED), impotence, female sexual dysfunction (FSD), clitoral dysfunction, female hypoactive sexual desire disorder, female sexual arousal disorder, female sexual pain disorder or female sexual orgasmic dysfunction (FSOD) as well as sexual dysfunction due to spinal cord injury or selective serotonin re-uptake inhibitor (SSRI) induced sexual dysfunction but, clearly, will be useful also for treating other medical conditions for which a cGMP PDE5 inhibitor is indicated. Such conditions include premature labour, dysmenorrhoea, benign prostatic hyperplasia (BPH), bladder outlet obstruction, incontinence, stable, unstable and variant (Prinzmetal) angina, hypertension, pulmonary hypertension, chronic obstructive pulmonary disease, coronary artery disease, congestive heart failure, atherosclerosis, conditions of reduced blood vessel patency, e.g. post-percutaneous transluminal coronary angioplasty (post-PTCA), peripheral vascular disease, stroke, nitrate induced tolerance, bronchitis, allergic asthma, chronic asthma, allergic rhinitis, diseases and conditions of the eye such as glaucoma, optic neuropathy, macular degeneration, elevated intra-occular pressure, retinal or arterial occulsion and diseases characterised by disorders of gut motility, e.g. irritable bowel syndrome (IBS).

Further medical conditions for which a potent and selective cGMP PDE5 inhibitor is indicated, and for which treatment with compounds of the present invention may be useful, include pre-eclampsia, Kawasaki's syndrome, nitrate tolerance, multiple sclerosis, diabetic nephropathy, neuropathy including autonomic and peripheral neuropathy and in particular diabetic neuropathy and symptoms thereof (e.g. gastroparesis), peripheral diabetic neuropathy, Alzheimer's disease, acute respiratory failure, psoriasis, skin necrosis, cancer, metastasis, baldness, nutcracker oesophagus, anal fissure, haemorrhoids, hypoxic vasoconstriction, hypoxic vasoconstriction, diabetes, type 2 diabetes mellitus, the insulin resistance syndrome, insulin resistance, impaired glucose tolerance, as well as the stabilisation of blood pressure during haemodialysis.

Particularly preferred conditions include MED and FSD.

Thus, the invention provides a method of treating or preventing a medical condition for which a cGMP PDE5 inhibitor is indicated, in an animal (e.g. a mammal, including a human being), which comprises administering a therapeutically effective amount of a compound of the invention to a mammal in need of such treatment.

Pharmaceutical Preparations

The compounds of the invention will normally be administered orally or by any parenteral route, in the form of pharmaceutical preparations comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

The compounds of the invention may also be combined with any other drugs useful in the inhibition of cGMP-PDEs, such as cGMP-PDE5.

The compounds of the invention, their pharmaceutically acceptable salts, and pharmaceutically acceptable solvates of either entity can be administered alone but, in human therapy will generally be administered in admixture with a suitable pharmaceutical excipient diluent or carrier selected with regard to the intended route of administration and standard pharmaceutical practice.

For example, the compounds of the invention or salts or solvates thereof can be administered orally, buccally or sublingually in the form of tablets, capsules (including soft gel capsules), ovules, elixirs, solutions or suspensions, which may contain flavouring or colouring agents, for immediate-, delayed-, modified-, or controlled-release such as sustained-, dual-, or pulsatile delivery applications. The compounds of the invention may also be administered via intracavernosal injection. The compounds of the invention may also be administered via fast dispersing or fast dissolving dosages forms.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intracavernosally, intravenously, intra-arterially, intraperitoneally, intrathecally, intraventricularly, intraurethrally intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention or salts or solvates thereof may contain from 5mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including MED and FSD), compounds of the invention may be taken as a single dose on an “as required” basis (i.e. as needed or desired).

Example Tablet Formulation

In general a tablet formulation could typically contain between about 0.01 mg and 500 mg of a compound according to the present invention (or a salt thereof) whilst tablet fill weights may range from 50 mg to 1000 mg. An example formulation for a 10 mg tablet is illustrated:

| Ingredient | % w/w |
|---|---|
| Compound of Example 1 | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity.

Such tablets can be manufactured by standard processes, for example, direct compression or a wet or dry granulation process. The tablet cores may be coated with appropriate overcoats.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark] or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention or salts or solvates thereof can be administered in the form of a suppository or pessary, or they may be applied topically in the form of a gel, hydrogel, lotion, solution, cream, ointment or dusting powder. The compounds of the invention or salts or solvates thereof may also be dermally administered. The compounds of the invention or salts or solvates thereof may also be transdermally administered, for example, by the use of a skin patch. They may also be administered by the ocular, pulmonary or rectal routes.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention or salts or solvates thereof can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration of the compounds of the invention is the preferred route, being the most convenient and, for example in MED, avoiding the well-known disadvantages associated with intracavernosal (i.c.) administration. A preferred oral dosing regimen in MED for a typical man is from 25 to 250 mg of compound when required. In circumstances where the recipient suffers from a swallowing disorder or from impairment of drug absorption after oral administration, the drug may be administered parenterally, sublingually or buccally.

For veterinary use, a compound of the invention, or a veterinarily acceptable salt thereof, or a veterinarily acceptable solvate or pro-drug thereof, is administered as a suitably acceptable formulation in accordance with normal veterinary practice and the veterinary surgeon will determine the dosing regimen and route of administration which will be most appropriate for a particular animal.

Thus, according to a further aspect of the invention there is provided a pharmaceutical formulation including a compound of the invention in admixture with a pharmaceutically or veterinarily acceptable adjuvant, diluent or carrier.

In addition to the fact that compounds of the invention inhibit cyclic guanosine 3′,5′-monophosphate phosphodiesterases (cGMP PDEs) and in particular, are inhibitors of cGMP PDE5, compounds of the invention may also have the advantage that they may be more efficacious than, be less toxic than, have a broader range of activity than, produce fewer side effects than, be more easily absorbed than, or they may have other useful pharmacological properties over, compounds known in the prior art.

Surprisingly, compound 1A is a pro-drug of the compound of Preparation 1. Compound 1A is converted in vivo to provide the compound of Preparation 1.

Furthermore, compound 1A has been shown to be a metabolite of the compound of Preparation 1. Compound 1A is formed from the compound of Preparation 1 in vivo.

Thus, according to a further aspect the present invention provides 5-[2-butoxy-5-(1-hydroxyethyl)-3-pyridinyl]-3-ethyl-2-(1-ethyl-3-azetidinyl)-2,6-dihydro-7H-pyrazolo[4,3-d]pyrimidin-7-one with the proviso that said compound is not obtained by metabolism of 5-(5-acetyl-2-butoxy-3-pyridinyl)-3-ethyl-2-(1-ethyl-3-azetidinyl )-2 ,6-dihydro-7H-pyrazolo[4,3-d]pyrimidin-7-one in the body.

According to a yet further aspect the present invention provides 5-[2-butoxy-5-( 1-hydroxyethyl)-3-pyridinyl]-3-ethyl-2-( 1-ethyl-3-azetidinyl)-2,6-dihydro-7H-pyrazolo[4,3-d]pyrimidin-7-one in isolated form.

Isolated form as defined herein means obtainable by chemical synthesis not by a biological process of the body, or part thereof.

According to a yet further aspect the present invention provides a compound of the formula 1A which is substantially pure.

According to another aspect the present invention provides the compound of preparation 1 when formed from compound 1A.

The biological activities of the compounds of the present invention were determined by the following test methods.

Phosphodiesterase (PDE) inhibitory activity

The compounds of the present invention are cGMP PDE5 inhibitors. In vitro PDE inhibitory activities against cyclic guanosine 3′,5′-monophosphate (CGMP) and cyclic adenosine 3′,5′-monophosphate (cAMP) phosphodiesterases were determined by measurement of their IC₅₀ values (the concentration of compound required for 50% inhibition of enzyme activity).

The required PDE enzymes were isolated from a variety of sources, including human corpus cavernosum, human and rabbit platelets, human cardiac ventricle, human skeletal muscle and human and canine retina, essentially by the method of W. J. Thompson and M. M. Appleman (Biochem., 1971, 10, 311). In particular, the cGMP-specific PDE (PDE5) and the cGMP-inhibited cAMP PDE (PDE3) were obtained from human corpus cavernosum or human platelets; the cGMP-stimulated PDE (PDE2) was obtained from human corpus cavernosum and human platelets; the calcium/calmodulin (Ca/CAM)-dependent PDE (PDE1) from human cardiac ventricle; the cAMP-specific PDE (PDE4) from human skeletal muscle and human recombinant, expressed in SF9 cells; and the photoreceptor PDE (PDE6) from human or canine retina. Phosphodiesterases 7-11 were generated from full length human recombinant clones transfected into SF9 cells.

Assays were performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc˜⅓ Kₘ) such that IC₅₀≅Kᵢ. The final assay volume was made up to 100 μl with assay buffer [20 mM Tris-HCl pH 7.4, 5 mM MgCl_{2, 1} mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension (or in-house equivalent). Results from these tests show that the compounds of the present invention are inhibitors of cGMP-specific PDE5.

Preferred compounds of the present invention have IC₅₀ values of less than about 1 μM for the PDE5 enzyme. A further preferred group of compounds have IC₅₀ values of less than about 0.9 μM for the PDE5 enzyme.

Highly preferred herein are compounds which have an IC₅₀ value of less than about 1 μM, more preferably less than about 0.9 μM for the PDE5 enzyme in combination with greater than 2-fold, preferably greater than 3-fold selectivity for the PDE5 enzyme versus the PDE6 enzyme.

Especially preferred herein is compound IA.

Biological Activity

Compounds of the invention were found to have in vitro activities as inhibitors of cGMP PDE5 with IC₅₀ values of less than about 1 μM.

The following Table illustrates the in vitro activities for compound 1A as an inhibitor of cGMP PDE5.

| Example | IC₅₀ (μM) |
|---|---|
| 1A | 0.825 |

Functional activity

This can be assessed in vitro by determining the capacity of a compound of the invention to enhance sodium nitroprusside or electrical field stimulation-induced relaxation of pre-contracted rabbit corpus cavernosum tissue strips, using methods based on that described by S. A. Ballard et al. (Brit. J. Pharmacol., 1996, 118 (suppl.), abstract 153P) or S. A. Ballard et al. (J. Urology, 1998, 159, 2164-2171).

In vivo activity

Compounds can be screened in anaesthetised dogs to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

Safety Profile

Compounds of the invention may be tested at varying i.v and p.o. doses in animals such as mouse, rat and dog, observing for any untoward effects.

Metabolism Data

Hepatocyte incubations were used to determine the metabolism of both compound 1A according to the present invention and also the compound of Preparation 1. In such testing a marker compound was routinely included to ensure reproducible, active preparations were obtained.

Standard methodologies relating to hepatocyte testing can be found in: Carlille, D, Zomorodi, K, Houston, B; Drug Met. Dispos. 1997, 25(8), pp903-911, “Scaling factors to relate drug metabolic clearance in hepatic microsomes isolated hepatocytes, and the intact liver”.

1. Materials used in Hepatocyte Incubation:

Marker compound: 0.3 mM Propranolol in 100% DMSO.

Test materials (e.g. compound 1A) 1 mg/ml stocks or 3 mM in 100% DMSO.

Human hepatocytes used at a concentration of 2×106 cells/ml (per compound tested).

Dog hepatocytes used at a concentration of 1×106 cells/ml (per compound tested).

William's E buffer (1 pot of William's E powder from Sigma, 900 ml distilled water, 2.2 g sodium bicarbonate, 100 ml Foetal Bovine Serum) STOP solution (Midazolam internal standard plus acetonitrile: 600 μl of 10 μg/ml midazolam +30 ml acetonitrile).

Trypan Blue solution to count cells/viability.

Krebs H buffer (made from 1 pot of powder with 1 L distilled water).

2. Hepatocyte Test Equipment:

50 ml centrifuge tubes; 96 deep well (2 ml) polypropylene blocks;

Centrifuge (IEC Centra GP8R); Multi-channel pipette (Biohit-Proline);

Haemocytometer (REICHERT, Buffalo, N.Y.); Polypropylene measuring cylinder; Plastic pasteur pipette.

3. Example Hepatocyte Test Method:

preparation of solutions of test compound(s)

10 μl of 3 mM test compound was added to 290 μl William's E buffer in a 96 well diluting block to provide a 100 μM stock solution of test compound.

20 μl of this stock solution was then added to 480 μl William's E buffer in an incubation block.

preparation of hepatocytes

50 ml centrifuge tubes were pre-cooled on ice (one tube per vial hepatocytes) and the centrifuge and buckets were also pre-cooled to 4° C. The hepatocytes for the test were stored in vials (1 ml vials containing ˜5×10⁶) in liquid nitrogen and once removed from said liquid nitrogen : (i) stored for 5 minutes on ice (ii) warmed at 37° C. in a water bath for 90 seconds with continuous swirling then (iii) placed back onto ice until preparation.

The defrosted hepatocytes were introduced to the centrifuge tubes by repeated, gentle washing of each vial with 12 ml Krebs H (KH) buffer. KH buffer is added dropwise followed by, careful transfer of the hepatocyte/KH mixture to the centrifuge tubes using a plastic pipette. During the buffer addition and transfer processes care should be taken to ensure bubble formation is minimised. Once all 12 ml has been used for washing and the resultant hepatocyte suspensions have been transferred to the centrifuge tube the washing process is complete. After gentle dispersion of the suspension of hepatocyte cells in the KH buffer the centrifuge tubes are then spun for 5 minutes at 500 rpm at 4° C.

After centrifugation the liquid supernatant was discarded and the hepatocyte pellet(s) were pooled and a further volume of KH buffer* was added. Where human hepatocyte cells were used in the test 1 ml KH buffer was added per original 1 ml vial of human hepatocyte cells and where dog hepatocyte cells were used in the test 2 ml KH buffer was added per 1 ml vial dog hepatocyte cells. The KH buffer solution was added dropwise, with gently swirling until the hepatocyte pellets were resuspended.

Hepatocyte viability was determined using the Trypan Blue Exclusion method by gentle mixing of 350 μl KH buffer+100 μI Trypan Blue solution+50 μl cell suspension in an eppendorf tube. The cells were then counted using a haemocytometer. As the haemocytometer is stored in 70% ethanol it was pre-washed with distilled water and blotted dry with tissue prior to use. The number of viable (colourless) cells within 10 squares was recorded. Cells that have been stained blue are considered to be non-viable. The calculation below was used to determine the cell concentration:

Cell number=(viable cells/10×10 000×10)/10⁶=x million viable cells/ml

[Where the number of squares counted=10, the haemocytometer factor=10,000 and the cell dilution is 1 in 10.]

The number of viable cells/ml was multiplied by the volume of buffer (KH*) initially used to determine the total number of viable cells. This number was then divided by the number of cells required. For each test compound the concentration of cells required for incubation is 4×106 for human and 2×106 cells for dog. This accounts for the dilution that occurs during incubation. Final hepatocyte suspension is adjusted accordingly with the addition of further KH buffer using an 8-tip multi-channel pipette

testing of compounds with hepatocytes

Prior to addition of the hepatocyte suspension the buffer/test compound mixtures had been pre-warmed in the 96 well block for 5 minutes in a water bath at 37° C. The reaction was initiated with the addition of 500 μl of the hepatocyte suspension (using an 8-tip multi-channel pipette) to the 96 well block containing the solution of test compound and William's E buffer. The final incubation volume was 1 ml with the test compound at a concentration of 2 μM. At timepoints of 0, 5, 15, 30, 60 90, 120, & 180 minutes 100 μl aliquots of incubate (hepatocytes +William's E buffer+test compound) were sampled into 200 μl STOP solution (e.g. 100% acetonitrile) in a separate 96 well block. After 3 hours the block containing the STOP solution was centrifuged for 1 hour at 3000 rpm, 4° C. After spinning the block was analysed via Turbulent flow liquid chromatography with tandem mass spectrometry detection. Further details relating to Turbulent-Flow Chromatography/Tandem Mass Spectrometry are found in WO 97/16724.

Results Obtained

Turbulent Flow LC-MS-MS, was used to monitor the mass transitions from the starting test compounds through to generation of the resultant compounds (metabolites).

A hepatocyte preparation of the compound of the ketone of Preparation 1 was subjected to metabolite identification testing and compound 1A of Example 1 was identified as the major metabolite. Further experiments were run to identify production of the alcohol from the ketone and vice versa as discussed below.

Results for the compound of Preparation 1 (the ketone). The mass transition corresponding to the starting compound (Preparation 1) of 439 to 84 reduces to levels below the detection limit of the equipment and then the mass ion with transition 441 to 84, corresponding to the alcohol (compound 1A), appears.

Results for the compound of Compound 1A (the alcohol). The mass ion transition corresponding to the starting compound (Compound 1A) at 441 to 84 reduces to levels below the detection limit of the equipment and then the mass ion with transition 439 to 84, corresponding to the ketone (preparation 1), appears.

Surprisingly the data generated illustrates that the ketone of preparation 1 is converted to the alcohol of 1A and vice versa. Compound 1A is a pro-drug of the compound of Preparation 1 and has been shown to be is converted in vivo to provide the compound of Preparation 1. Furthermore, Compound 1A is a metabolite of the compound of Preparation 1 and has been shown to be formed from the compound of Preparation 1 in vivo.

### EXAMPLES AND PREPARATIONS

The synthesis of the compounds of the invention and of the intermediates for use therein are illustrated by the following Examples and Preparations.

¹H nuclear magnetic resonance (NMR) spectra were recorded using either a Varian Unity 300 or a Varian Inova 400 spectrometer and were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad.

Mass spectra (m/z) were recorded using a Fisons Instruments Trio mass spectrometer in the thermospray ionisation mode (TSP) or using a Finnigan navigator in electrospray ionisation mode (ES)—positive and/or negative ionisation mode.

As used herein, the term “column chromatography” refers to normal phase chromatography using silica gel (0.04-0.06 mm).

Room temperature includes 20 to 25° C.

Synthesis of Compound 1A

### Example 1

### 5-[2-Butoxy-5-(1-hydroxyethyl )-3-pyridinyl]-3-ethyl-2-(1-ethyl-3-azetidinyl)-2,6-dihydro-7H-pyrazolo[4,3-d]pyrimidin-7-one

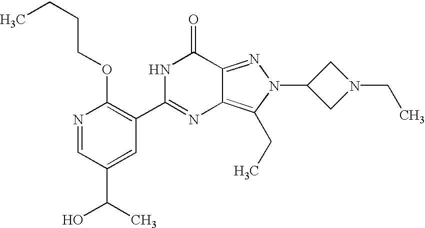

Sodium borohydride (17 mg, 0.46 mmol) was added to an ice-cooled suspension of 5-[5-acetyl-2-butoxy-3-pyridinyl]-3-ethyl-2-(1-ethyl-3-azetidinyl)-2,6-dihydro-7H-pyrazolo[4,3-d]pyrimidin-7-one (400 mg, 0.91 mmol) in methanol (10 ml) under a nitrogen atmosphere, and the solution allowed to warm to room temperature, with stirring, over 1 hour. Tlc analysis showed starting material remaining, so additional sodium borohydride (17 mg, 0.46 mmol) was added, and the reaction stirred at room temperature for 30 minutes. The mixture was concentrated under reduced pressure and the residue partitioned between ethyl acetate (30 ml) and water (20 ml), and the layers separated. The aqueous phase was extracted with ethyl acetate (2×20 ml), and the combined organic solutions were dried (MgSO₄) and evaporated under reduced pressure. The residual yellow foam was purified by column chromatography on silica gel using dichloromethane:methanol:0.88 ammonia (95:5:0.5) as eluant. The resulting foam was crystallised from diethyl ether to afford the title compound as a white solid, (285 mg, 0.65 mmol).

m.p. 117-119° C.

¹Hnmr (CDCl₃, 400 MHz) δ: 1.02 (m, 6H), 1.34 (t, 3H), 1.52 (m, 2H), 1.59 (d, 3H), 1.90 (m, 2H), 2.28 (bs, 1H), 2.66 (q, 2H), 3.00 (q, 2H), 3.73 (t, 2H), 3.90 (t, 2H), 4.54 (t, 2H), 5.00 (m, 1H), 5.13 (m, 1H), 8.23 (s, 1H), 8.72 (s, 1H), 10.83 (bs, 1H).

LRMS: m/z (TSP+) 441.2 (MH+)

Microanalysis found: C, 62.45; H, 7.35; N, 18.85. C₂₃H₃₂N₆O₃ requires C, 62.71; H, 7.32; N, 19.08%.

### Preparation 1

5-(5-acetyl-2-butoxy-3-pyridinyl)-3-ethyl-2-(1-ethyl-3-azetidinyl)-2,6-dihydro-7H-pyrazolo[4,3-d]pyrimidin-7-one.

5-(5-Acetyl-2-propoxy-3-pyridinyl)-3-ethyl-2-(1-ethyl-3-azetid inyl)-2,6-dihydro-7H-pyrazolo[4,3-d]pyrimidin-7-one (120 mg, 0.28 mmol) and cesium carbonate (274 mg, 0.84 mmol) were dissolved in n-butanol (4 ml), and heated at 90° C. under nitrogen with molecular sieves for 96 h. The mixture was then partitioned between water (10 ml) and dichloromethane (10 ml). The organic layer was separated, and the aqueous layer extracted further with dichloromethane (3×15 ml). The combined organic layers were dried (MgSO₄), and concentrated in vacuo. The crude product was purified by flash column chromatography (95:5:0.5-90:10:1 ethyl acetate:methanol:0.88 NH₃ as eluents), to yield the title compound as a colourless glass (77 mg, 0.18 mmol).

m.p. 91.6-93.7° C.

1H NMR (400 MHz, CDCl₃): δ=1.00-1.05 (m, 6H), 1.38 (t, 3H), 1.50-1.62 (m, 2H), 1.90-2.00 (m, 2H), 2.63 (s, 3H), 2.63-2.70 (m, 2H), 3.02 (q, 2H), 3.75 (t, 2H), 3.90 (t, 2H), 4.68 (t, 2H), 5.10-5.20 (m, 1H), 8.84 (s, 1H), 9.23 (s, 1H), 10.63 (brs, 1H).

LRMS (TSP—positive ion) 439 (MH+)

Anal. Found C, 60.73; H, 7.06; N, 18.03 Calcd for C₂₃H₃₀O₃N₆.0.2MeOH.0.1 DIPE: C, 60.88; H, 7.26; N, 17.90

Preparation of starting materials for Example 1A

### Preparation 1

5-(5-acetyl-2-butoxy-3-pyridinyl)-3-ethyl-2-(1-ethyl-3-azetidinyl)-2,6-dihydro-7H-pyrazolo[4,3-d]pyrimidin-7-one.

The title compound from preparation 4(a) (120 mg, 0.28 mmol) and cesium carbonate (274 mg, 0.84 mmol) were dissolved in n-butanol (4 ml), and heated at 90° C. under nitrogen with molecular sieves for 96 h. The mixture was then partitioned between water (10 ml) and dichloromethane (10 ml). The organic layer was separated, and the aqueous layer extracted further with dichloromethane (3×15 ml). The combined organic layers were dried (MgSO₄), and concentrated in vacuo. The crude product was purified by flash column chromatography (95:5:0.5-90:10:1 ethyl acetate:methanol:0.88 NH₃ as eluents), to yield the title compound as a colourless glass (77 mg, 0.18 mmol).

m.p. 91.6.93.7° C.

1H NMR (400 MHz, CDCl₃): δ=1.00-1.05 (m, 6H), 1.38 (t, 3H), 1.50-1.62 (m, 2H), 1.90-2.00 (m, 2H), 2.63 (s, 3H), 2.63-2.70 (m, 2H), 3.02 (q, 2H), 3.75 (t, 2H), 3.90 (t, 2H), 4.68 (t, 2H), 5.10-5.20 (m, 1H), 8.84 (s, 1H), 9.23 (s, 1H), 10.63 (br s, 1H).

LRMS (TSP—positive ion) 439 (MH+)

Anal. Found C, 60.73; H, 7.06; N, 18.03 Calcd for C₂₃H₃₀O₃N₆.0.2MeOH.0.1 DIPE: C, 60.88; H, 7.26; N, 17.90

Preparation of starting materials for Example 1

1(a) 5-(5-Acetyl-2-propoxy-3-pyridinyl)-3-ethyl-2-(1-ethyl-3-azetidinyl)-2,6-dihydro-7H-pyrazolo[4,3-d]pyrimidin-7-one

Sodium cyanoborohydride (92 mg, 1.47 mmol) was added to a stirring solution of title compound from example 1(b) (500 mg, 0.98 mmol), acetaldehyde (64 μl, 1.18 mmol) and sodium acetate (161 mg, 1.96 mmol) in methanol (10 ml) under nitrogen at room temperature. After 1 h the mixture was poured into NaHCO₃ (sat. aq., 20 ml), and extracted with dichloromethane (3×15 ml). The combined organic layers were dried (MgSO₄) and concentrated in vacuo. The crude product was purified by flash column chromatography (95:5:0.5-80:20:1 ethyl acetate:methanol:0.88 NH₃ as eluent) to yield the title compound as a white solid (140 mg, 0.33 mmol).

1H NMR (400 MHz, CDCl₃): δ=0.97 (t, 3H), 1.03 (t, 3H), 1.30 (t, 3H), 2.82-2.97 (m, 2H), 2.58-2.65 (m, 5H), 2.98 (q, 2H), 3.68 (t, 2H), 3.85 (dd, 2H), 4.58 (dd, 2H), 5.05-5.17 (m, 1H), 8.79 (s, 1H), 9.18 (s, 1H), 10.62 (br s, 1H).

LRMS (TSP—positive ion) 426 (MH+)

1(b) 5-(5-Acetyl-2-propoxy-3-pyridinyl)-2-(3-azetidinyl)-3-ethyl-2,6-dihydro-7H-pyrazolo[4,3-d]pyrimidin-7-one

The title compound of Preparation 1(c) (1.44 g, 3.0 mmol) in acetone (50 ml) and sulphuric acid (1N, 3 ml) was treated with mercuric sulphate (268 mg, 9.0 mmol) and heated to reflux for 6 h. The reaction mixture was concentrated to ˜20 ml in vacuo, poured into sodium bicarbonate (sat. aq., 20ml) and extracted into methylene chloride (6×20 ml). Combined organics were washed with brine (20 ml), dried over MgSO₄, and concentrated to a brown oil which was taken up in 40% trifluoroacetic acid in methylene chloride (50 ml) and water (1 ml) and stirred for 1 h at room temperature. After evaporation in vacuo, the residue was purified by column chromatography (eluting with 95:5:1 methylene chloride:methanol:0.88 ammonia) to afford the title compound as a white hydroscopic foam (1.65 g).

m.p. 128.5-130.0° C.

1H NMR (400 MHz, MeOD): δ=1.00 (t, 3H), 1.30 (t, 3H), 1.79-1.90 (m, 2H), 2.60 (s, 3H), 3.00-3.10 (q, 2H), 4.50 (t, 2H), 4.60-4.70 (m, 4H), 5.65-5.78 (m, 1H), 8.65 (s, 1H), 8.90 (s, 1H)

LRMS (TSP—positive ion) 397 (MH+)

1(c) tert-Butyl 3-[3-ethyl-5-(5-ethynyl-2-propoxy-3-pyridinyl)-7-oxo-6,7-dihydro-2H-pyrazolo[4,3-d]pyrimidin-2-yl]-1 -azetidinecarboxylate

Prepared from the title compound of Preparation 1(d) by the method of Preparation 1(c)(i).

1H NMR (400 MHz, CDCl₃): δ=1.05 (t, 3H), 1.30 (t, 3H), 1.43 (s, 9H), 1.88-2.00 (m, 2H), 3.00 (q, 2H), 3.19 (s, 1H), 4.35 (app t, 2H), 4.52 (app t, 2H), 4.60-4.80 (br s, 2H), 5.22 (t, 1H), 8.39 (s, 1H), 8.80 (s, 1H), 10.75 (br s, 1H)

LRMS (TSP—positive ion) 496 (MNH₄+).

1(c)(i) 5-(2-Butoxy-5-ethynyl-3-pyridinyl)-3-ethyl-2-(2-methoxyethyl)-2,6-dihydro-7H-pyrazolo[4,3-d]pyrimidin-7-one

Potassium fluoride (22 mg, 0.38 mmol) was added to a stirred solution of the title compound of Preparation 1(d)(i) (90 mg, 0.19 mmol) in aqueous N,N-dimethylformamide (2 mL N,N-dimethylformamide /0.2 mL water) at 0° C. After 10 min the reaction was allowed to warm to room temperature and stirred for 2 h. The reaction mixture was diluted with ethyl acetate and washed with water, 1N hydrochloric acid (3 times) and brine. The organic layer was dried (MgSO₄) and concentrated to give the title compound as a white solid (75 mg).

¹H NMR (400 MHz, CDCl₃): δ=1.00 (t, 3H), 1.40 (t, 3H), 1.50 (m, 2H), 1.90 (m, 2H), 3.05 (q, 2H), 3.20 (s, 1H), 3.30 (s, 3H), 3.85 (t, 2H), 4.40 (t, 2H), 4.60 (t, 2H), 8.40 (s, 1H), 8.80 (s, 1H), 10.70 (s, 1H).

LRMS (TSP): 396.3 (MH+).

1(d) tert-Butyl 3-(3-ethyl-7-oxo-5-{2-propoxy-5-[(trimethylsilyl)ethynyl]-3-pyridinyl}-6,7-dihydro-2H-pyrazolo[4,3-d]pyrimidin-2-yl)-1-azetidinecarboxylate

Prepared from the title compound of Preparation 1(e) by the method of Preparation 1(d)(i).

1H NMR (400 MHz, MeOD): δ=0.25 (s, 9H), 1.05 (t, 3H), 1.31 (t, 3H), 1.44 (s, 9H), 1.87-1.96 (m, 2H), 3.00 (q, 2H), 4.33 (t, 2H), 4.52 (t, 2H), 4.54-4.80 (m, 2H), 5.18-5.25 (m, 1H), 8.32 (d, 1H), 8.74 (d, 1H)

LRMS (TSP—positive ion) 569 (MNH₄+), 552.0 (MH+)

Anal. Found C, 60.82; H, 6.90; N, 15.15 Calcd for C₂₈H₃₈O₄N₆Si: C, 61.07; H, 6.95; N, 15.26.

1(d)(i) 5-(2-Butoxy-5-trimethylsilylethynyl-3-pyridinyl)-3-ethyl-2-(2-methoxy-ethyl)-2,6-dihydro-7H-pyrazolo[4,3-d]pyrimidin-7-one

The title compound from Example 1 of PCT application WO 01/27112 (127 mg, 0.25 mmol) was suspended in triethylamine (2 mL) and trimethylsilylacetylene (38 mg, 0.39 mmol) and acetonitrile (2 mL). Pd(PPh₃)₂Cl₂ (5 mg, 0.006 mmol) and cuprous iodide (1.2 mg, 0.006 mmol) were added and the reaction mixture stirred. After 1 h a further portion of trimethylsilylacetylene (19 mg, 0.19 mmol) was added and stirring continued for 2 h. The solvent was evaporated and the residue partitioned between ethyl acetate and water. The organics were washed with brine, dried (MgSO₄) and concentrated to give a brown foam. Purification by flash column chromatography (gradient elution from 100% dichloromethane to 99% dichloromethane/methanol) gave the title compound as a light brown solid (108 mg).

¹H NMR (300 MHz, CDCl₃): δ=0.25 (s, 9H), 1.00 (t, 3H), 1.40 (t, 3H), 1.50 (m, 2H), 1.90 (m, 2H), 3.10 (q, 2H), 3.30 (s, 3H), 3.90 (t, 2H), 4.40 (t, 2H), 4.60 (t, 2H), 8.40 (s, 1H), 8.80 (s, 1H), 10.70 (s, 1H).

LRMS (TSP): 468.3 (MH+).

1(e) tert-Butyl 3-[3-ethyl-5-(5-iodo-2-propoxy-3-pyridinyl)-7-oxo-6,7-dihydro-2H-pyrazolo[4,3-d]pyrimidin-2-yl]-1 -azetidinecarboxylate

The title compound was prepared from the product of Preparation 1(f) using the method of Preparation 1(e)(i).

1H NMR (400 MHz, CDCl₃): δ=1.05 (t, 3H), 1.30 (t, 3H), 1.43 (s, 9H), 1.87-1.96 (m, 2H), 3.00 (q, 2H), 4.34 (t, 2H), 4.49 (t, 2H), 4.60 (br s, 2H), 5.20 (t, 1H), 8.41 (d, 1H), 8.94 (s, 1H), 10.75 (brs, 1H)

LRMS (TSP—positive ion) 598.1 (MNH₄+)

Anal. Found C, 47.54; H, 5.02; N, 14.09 Calcd for C₂₃H₂₉O₄N₆I: C, 47.60; H, 5.04; N, 14.48.

4(e)(i) 3-Ethyl-5-(5-iodo-2-propoxy-3-pyridinyl)-1-[2-(4-morpholinyl)ethyl]-1,6-dihydro-7H-pyrazolo[4,3-d]pyrimidin-7-one

The title compound of Preparation 48 of PCT application WO 01/27112 (15.78 g, 28.4 mmol) was dissolved in n-propanol (200 ml), ethyl acetate (6 ml) and potassium t-butoxide (3.2 g, 28.4 mmol) were added and the resultant mixture heated to reflux for 6 h. Additional potassium t-butoxide (1.6 g, 14.2 mmol) was added and the mixture heated for a further 2 h, after which the solvent was removed in vacuo. The residue was partitioned between water (50 ml) and methylene chloride (100 ml) and the organic phase separated. The aqueous phase was extracted with dichloromethane (2×100 ml) and the combined organics dried over MgSO₄ and reduced to a yellow solid (˜17 g). Purification by column chromatography (elution with ethyl acetate) gave the title compound (13.3 g, 24.1 mmol) together with recovered starting material (2.31 g, 4.2 mmol).

m.p. 175-177° C.

1H NMR (300 MHz, CDCl₃): δ=1.1 (t, 3H), 1.4 (t, 3H), 1.9-2.05 (m, 2H), 2.45-2.55 (m, 4H), 2.85 (t, 2H), 3.0 (q, 2H), 3.6-3.65 (m, 4H), 4.5 (t, 2H), 4.7 (t, 2H), 8.4 (s, 1H), 9.0 (s, 1H), 10.95 (br s, 1H).

LRMS (TSP) 540 (MH+).

Analysis: found C, 46.79; H, 5.01; N, 15.44. Calcd for C₂₁H₂₇N₆O₃I: C, 46.85; H, 5.05; N, 15.61%

1(f) tert-Butyl 3-(3-(aminocarbonyl)-5-ethyl-4-{[(5-iodo-2-propoxy-3-pyridinyl)carbonyl]amino}-1H-pyrazol-1-yl)-1-azetidinecarboxylate

The title compound was prepared by the method of Preparation 1(f)(i) using the products from Preparations 1(g) and 1(i).

1H NMR (400 MHz, DMSO): δ=0.95 (t, 3H), 1.05 (t, 3H), 1.40 (s, 9H), 1.78-1.88 (m, 2H), 2.68 (q, 2H), 4.22-4.35 (m, 4H), 4.40 (t, 2H), 5.33 (t, 1H), 7.35 (bs, 1H), 7.52 (bs, 1H), 8.40 (s, 1H), 8.55 (s, 1H), 10.10 (s, 1H)

LRMS (TSP—positive ion) 373.2 (MH+-BOC and I)

Anal. Found C, 45.11; H, 5.07; N, 13.56 Calcd for C₂₃H₃₁O₅N₆I. 0.2 DCM: C, 45.28; H, 5.14; N, 13.66.

1(f)(i) N-{3-(Aminocarbonyl)-1-[2-dimethylamino)ethyl]-5-ethyl-1H-pyrazol-4-yl}-2-butoxy-5-iodonicotinamide

Cesium carbonate (1.17 g, 3.59 mmol) was added to a stirred solution of the title compound from Preparation 16 of PCT application WO 01/27112 (800 mg, 1.79 mmol) and N,N-dimethylaminoethyl chloride hydrochloride (309 mg, 2.15 mmol) in N,N-dimethylformamide (10 mL) under a nitrogen atmosphere. The mixture was heated at 80° C. for 24 h. The mixture was cooled and extracted from water with ethyl acetate. The organics were dried (MgSO₄) and concentrated to give a brown oil. Purification by flash column chromatography (gradient elution from 100% dichloromethane to 90% dichloromethane/MeOH) gave the product as a pale brown oil (522 mg).

¹H NMR (400 MHz, CDCl₃): δ=0.95 (t, 3H), 1.20 (t, 3H), 1.40 (m, 2H), 1.90 (m, 2H), 2.35 (s, 6H), 2.80 (t, 2H), 2.85 (q, 2H), 4.20 (t, 2H), 4.60 (t, 2H), 5.30 (br s, 1H), 6.60 (br s, 1H), 8.40 (s, 1H), 8.75 (s, 1H), 10.35 (s, 1H).

LRMS (TSP): 529.5 (MH+).

1(g) N-[3-(Aminocarbonyl)-5-ethyl-1H-pyrazol-4-yl]-5-iodo-2-propoxy-nicotinamide

The title compound was prepared from 2-propoxy-5-iodonicotinic acid (see Preparation 1(h) and 4-amino-3-ethyl-1H-pyrazole-5-carboxamide (prepared as described in WO 98/49166) according to the method described in Preparation 1(g)(i).

¹H NMR (300 MHz, d₄-MeOH): δ=1.0 (t, 3H), 1.25 (t, 3H), 1.85-2.0 (m, 2H), 2.8 (q, 2H), 4.5 (t, 2H), 8.5 (s, I H), 8.6 (s, 1H).

LRMS (TSP) 444 (MH+).

1(g)(i) N-[3-(Aminocarbonyl)-5-ethyl-1-(2-methoxyethyl)-1H-pyrazol-4-yl]-2-butoxy-5-iodonicotinamide

Oxalyl chloride (2 g, 15.9 mmol) was added to a stirred solution of the title compound from Preparation 4 of PCT application WO 01/27112 (1.28 g, 3.98 mmol) in dichloromethane (20 mL) and, 3 drops N,N-dimethylformamide added. After 2.5 h the solvent was evaporated and the residue azeotroped 3 times with dichloromethane. The residue was resuspended in dichloromethane (4 mL) and added to a stirred mixture of the title compound of Preparation 11 from PCT application WO 01/27112 (0.76 g, 3.58 mmol) and triethylamine (0.8 g, 7.97 mmol) in dichloromethane (10 mL). After 1 h the solvent was evaporated and the residue partitioned between ethyl acetate and water. The organic phase was separated and washed with 2N HCl (twice), sodium bicarbonate solution (twice) and brine before being dried (MgSO₄) and concentrated. The product was triturated with ether and filtered to give 820 mg of pure product as a white solid. The mother liquor was concentrated and purified by flash column chromatography (elution with 80% ethyl acetate: hexane), to give a further 605 mg of product.

¹H NMR (400 MHz, CDCl₃): δ=0.95 (t, 3H), 1.20 (t, 3H), 1.45 (m, 2H), 1.90 (m, 2H), 2.85 (q, 2H), 3.35 (s, 3H), 3.80 (t, 2H), 4.25 (t, 2H), 4.60 (t, 2H), 5.20 (br s, 1H), 6.60 (br s, 1H), 8.40 (s, 1H), 8.80 (s, 1H), 10.30 (s, 1H).

LRMS (TSP): 516.2 (MH+).

1(h) 2-Propoxy-5-iodonicotinic acid

The title compound was prepared from 2-propoxy nicotinic acid (prepared as described in WO 99/54333, the compound 2-n-propoxypyridine-3-carboxylic acid, Preparation 46 prepared by the process of Preparation 1) using the method of Preparation 1(h)(i).

¹H NMR (300 MHz, CDCl₃): 6=1.05 (t, 3H), 1.85-2.0 (m, 2H), 4.5 (t, 2H), 8.5 (s, 1H), 8.6 (s, 1H).

Analysis: found C, 35.16; H, 3.19; N, 4.46. Calcd for CgH₁₀INO₃: C, 35.19; H, 3.28; N, 4.56%

1(h)(i) 2-isoButoxy-5-iodo nicotinic acid

N-lodosuccinamide (18.22 g, 0.08 mol), trifluoroacetic acid (100 mL) and trifluoroacetic anhydride (25 mL) were added to 2-isobutoxynicotinic acid (10.55 g, 0.054 mol). The mixture was refluxed for 2.5 h, cooled and the solvents evaporated. The residue was extracted from water with ethyl acetate and the organics washed with water (twice) and brine (twice), dried (MgSO₄) and concentrated. The red residue was redissolved in ethyl acetate washed with sodium thiosulfate solution (twice), water (twice), brine (twice), redried (MgSO₄) and concentrated to give the desired product as a yellow solid.

¹H NMR (300 MHz, CDCl₃): δ=1.05 (d, 6H), 2.20 (m, 1H), 4.40 (d, 2H), 8.50 (s, 1H), 8.70 (s, 1H),

LRMS (TSP): 322.3 (MH+).

1(i) tert-Butyl 3-iodo-1-azetidinecarboxylate

A mixture of tert-butyl 3-[(methylsulfonyl)oxy]-1-azetidinecarboxylate (prepared as described in Synlett 1998, 379; 5.0 g, 19.9 mmol), and potassium iodide (16.5 g, 99.4 mmol) in N,N-dimethylformamide (25 mL), was heated at 100° C. for 42 h. The cooled mixture was partitioned between water and ethyl acetate, and the layers separated. The organic phase was dried over MgSO₄, concentrated under reduced pressure and the residue azeotroped with xylene. The crude product was purified by flash column chromatography (dichloromethane as eluant) to give the title compound, 3.26 g.

¹H NMR (300 MHz, CDCl₃) δ=1.43 (s, 9H), 4.28 (m, 2H), 4.46 (m, 1H), 4.62 (m, 2H).

LRMS (TSP) 284 (MH)+

1. A compound of general formula I: 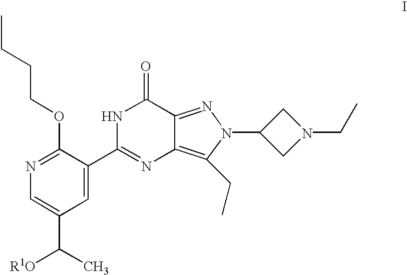
or a pharmaceutically or veterinarily acceptable salt or polymorph and/or solvate thereof, wherein
R¹ represents H; C(O)C₁-C₄ alkyl; C(O)aryl; C(O)heteroaryl.

2. A compound according to claim 1, wherein R¹ represents H or C(O)C₁-C₃ alkyl.

3. A compound according to claim 1 or 2, wherein R¹ is H or C(O)CH₃.

4. A compound according to claim 1 which is 5-[2-butoxy-5-(1-hydroxyethyl)-3-pyridinyl]-3-ethyl-2-(1-ethyl-3-azetidinyl)-2,6-dihydro-7H-pyrazolo[4,3-d]pyrimidin-7-one.

5. A compound according to claim 1 which is 5-[2-butoxy-5-(1-hydroxyethyl)-3-pyridinyl]-3-ethyl-2-(1-ethyl-3-azetidinyl)-2,6-dihydro-7H-pyrazolo[4,3-d]pyrimidin-7-one with the proviso that said compound is not obtained by metabolism of 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one in the body.

6. A compound as defined in any one of claims 1 to 5 for use as a pharmaceutical.

7. A compound as defined in any one of claims 1 to 5 for use as an animal medicament.

8. A formulation comprising a compound as defined in any one of claims 1 to 5 in admixture with a pharmaceutically or veterinarily acceptable adjuvant, diluent or carrier.

9. A formulation as claimed in claim 8, which is a pharmaceutical formulation.

10. A formulation as claimed in claim 8, which is a veterinary formulation.

11. The use of a compound as defined in any one of claims 1 to 5 in the manufacture of a medicament for the curative or prophylactic treatment of a medical condition for which inhibition of cGMP PDE5 is desired.

12. A method of treating or preventing a medical condition for which inhibition of cGMP PDE5 is desired, which comprises administering a therapeutically effective amount of a compound as claimed in any one of claims 1 to 5 to a patient in need of such treatment.

13. Use as claimed in claim 11, or method as claimed in claim 12, wherein the condition is male erectile dysfunction (MED), impotence, female sexual dysfunction (FSD), clitoral dysfunction, female hypoactive sexual desire disorder, female sexual arousal disorder, female sexual pain disorder or female sexual orgasmic dysfunction (FSOD).
